(19) United States
(12) Patent Application Publication
(10) Pub. No.: US 2004/0153149 A1
(43) Pub. Date: Aug. 5, 2004

(54) Intra-ocular implant

(76) Inventors: John L. Ricci, Middletown, NJ (US); Harold Alexander, Short Hills, NJ (US)

Correspondence Address: MELVIN K. SILVERMAN, SUITE 500, 500 WEST CYPRESS CREEK ROAD, FORT LAUDERDALE, FL, 33309, US

(21) Appl. No.: 10/241,256

(22) Filed: Sep. 10, 2002

Related U.S. Application Data
(60) Provisional application No. 60/317,830, filed on Sep. 10, 2001.

(51) Int. Cl. A61F 2/14

(52) U.S. Cl. 623/5.15

(57) ABSTRACT

An intra-ocular implant exhibits an interior and a posterior surface and includes an arcuate core element capable of optically transmitting light from an exterior of an eye to its retina, the core formed of an alloplastic material. An arcuate first skirt extends integrally and annularly about the optical core. An outer skirt extends integrally and annularly about the first skirt in which the outer skirt is formed of an hydrophilic material capable of receiving vascular invasion and formation of fibrous tissue. The outer skirt is characterized by a substantially flat posterior surface furnished with microgrooves having respective widths in a range of between about two to about twenty micrometers and a depth within a like range of micrometers.

### REFERENCE TO RELATED APPLICATION

This application is non-provisional conversion of provisional Application Serial No. 60/317,830, filed Sep. 10, 2001. The benefit thereof is claimed, and the same is hereby incorporated by reference, under 35 U.S.C. 119(e).

N/A.

### BACKGROUND OF THE INVENTION

Intra-ocular implants, typically in the form of a corneal prosthesis, are known in the art, as is reflected in such references as U.S. Pat. No. 5,489,301 (1996) to Barber, entitled Corneal Prosthesis; and U.S. Pat. No. 6,106,552 (2000) to Lacombe, et al, entitled Corneal Prosthesis Device Having Anterior and Posterior Annular Skirts.

Such intra-ocular implants address a range of opthalmatic issues, including aphakia (the absence of a lens).

Historic problems in the use of intra-ocular implants in apikaratoplasty or epikeiratophakia have been that of assuring the stability of the implant relative to the region of the eye upon which it is to be secured, and the prevention of epithelial overgrowth thereby causing opacity. As is apparent, any movement or dislocation of an intra ocular implant from its desired placement can have consequences which are at least adverse and often disastrous in terms of the success of a given procedure. Likewise, opacity due to overgrowth can severely compromise the success of the implant procedure.

The present invention is therefore directed to an intra-ocular implant having improved means of securement thereof to appropriate surrounding tissue of the human eye and a means of directing tissue growth away from the visual areas.

### SUMMARY OF THE INVENTION

The instant intra-ocular implant exhibits an interior and a posterior surface and includes an arcuate core element capable of optically transmitting light from an exterior of an eye to a retina thereof, said core formed of an alloplastic material. An arcuate first skirt extends integrally and annularly about said optical core. An outer skirt extends integrally and annularly about said first skirt in which said outer skirt is formed of an hydrophilic material capable of receiving vascular invasion and formation of fibrous tissue therein. Said outer skirt is characterized by a substantially flat posterior surface furnished with a multiplicity of microgrooves having respective widths in a range of between about two to about twenty micrometers and a depth within a like range of micrometers. Such an implant will typically take the form of a corneal or apiketophakia prosthesis.

It is therefore an object of the present invention to provide an intra-ocular implant having enhanced stability within the human eye and a means of preventing epithelial overgrowth.

It is another object to provide an intra-ocular implant of the above type having improved co-action with vascular and fibrous tissue placed in contact with which an annulus of a posterior surface of the implant following corneal implant surgery.

It is a further object of the invention to provide an ocular implant of the above type having application in various post operative and traumatic conditions of the eye.

The above and yet other objects and advantages of the invention will become apparent from the hereinafter-set forth Brief Description of the Drawings and Detailed Description of the Invention and claims appended herewith.

### BRIEF DESCRIPTION OF THE DRAWINGS

FIG. 1 is a front elevational view of an intra-ocular implant in accordance with the invention.

FIG. 2 is a diametric cross-sectional view taken along Line 2-2 of FIG. 1.

FIG. 3 is a radial view of an annular segment of the implant taken along Line 3-3 of FIG. 2.

FIG. 4 is an enlarged schematic view of the posterior surface of the outer skirt of the implant showing its interaction with vascular and fibrous tissue with which it is in contact after implantation.

### DETAILED DESCRIPTION OF THE INVENTION

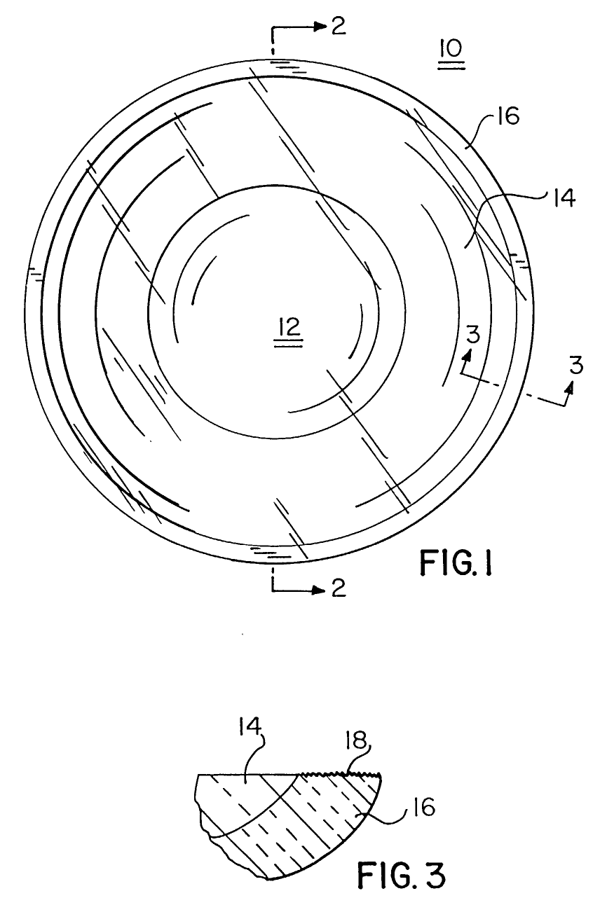

With reference to the front elevational view of FIG. 1, a preferred embodiment of the intra-ocular implant comprises a corneal prosthesis 10 having an arcuate optical core element capable of optically transmitting from an exterior of an eye to the retina thereof. Said core is formed of an alloplastic or other suitable polymeric material.

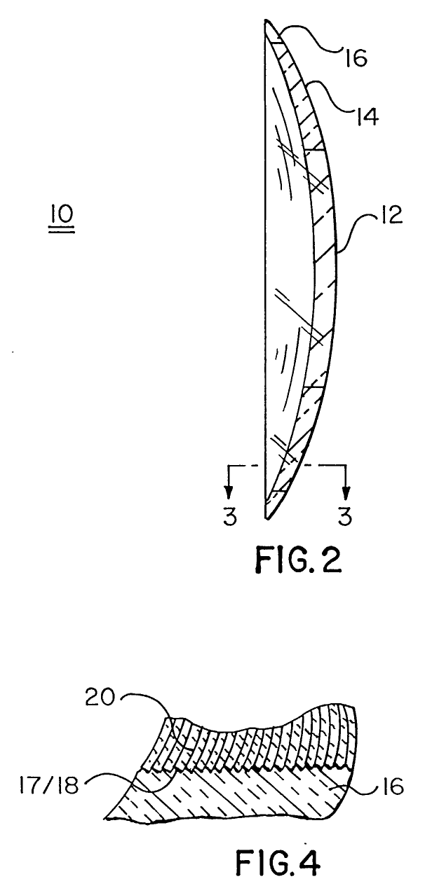

With further reference to FIGS. 1 and 2, the implant may be seen to further include a first arcuate skirt 14 extending integrally and annularly about said optical core 12. The material of the first skirt will typically be similar in type, however, somewhat greater in density than that of optical core element 12, given that its principal function is that of support and definition of the structure of the implant, while that of the optical core element 12 is that of transmission of light from an exterior of the eye to the retina.

At the periphery of the first arcuate skirt 14 is provided an outer skirt 16 which extends integrally and annularly about said first skirt. Skirt 14 is preferably formed of a hydrophilic material which is both biocompatible with the tissue of the human eye and capable of receiving vascular invasion and formation of fibrous tissue therein during the healing process. Materials presently known in the art that are suitable for formation of said first skirt 12 and outer skirt 16 include, without limitation, semi-flexible materials such as acrylate/methacrylate copolymers, silicon elastomers and porous polytetraflouroethylene.

A posterior contact surface 17 of outer skirt 16 (see FIG. 3) is typically at least 100 square millimeters, with a suitable flexure modulus. When the prosthesis is implanted in the eye, the radius of outer skirt 16 typically falls within a range of two to ten millimeters. Such a width is necessary, particularly, for a corneal prosthesis, to assure sufficient structural integrity upon which to apply the below described multiplicity of microgrooves. The width of outer skirt 16 is typically in a range of 0.5 to 1.0 millimeters. As such, it comprises a very thin, however, relatively wide element relative to other dimensions in the environment of the eye.

It is noted that the polymeric material of optical core element 12 and first arcuate skirt 14 need not be biocolonizable, inasmuch as core element 12 and first skirt 14 will not be required to support tissue growth. However, outer skirt 16 must be formed of a material having demonstrative properties of biocompatibility and must be biocolonizable, while having sufficient density and flexibility for purposes of long-term integration into human tissue. The above set forth polytetrafluoroethylene, a/m copolymers and silicon elastomers has been found to be suitable materials.

For production of the optical core element 12, an intra ocular grade of a/m copolymer has been found to comprise an acceptable material.

In the cross-sectional FIG. 3 is shown a radial fragment of the posterior surface 17 of the ocular implant and, together therewith, microgrooving 18 which, as is schematically shown in FIG. 4 facilitates the vascular invasion of tissue 20 typically received from the lamellar pocket of the eye, as well as the formation, over time, of fibrous tissue from tissue 20 into said microgrooves. Thereby, said multiplicity of microgrooves 18, having respective widths in a range of between 2 to about 20 micrometers and depths within a like range of micrometers, facilitate long term stability of the implant 10 relative to tissue 20 to which it is secured. In a preferred embodiment, microgrooves having a width and depth of about 8 millimeters have proven operative, with a range in width and depth of about 4 to about 12 micrometers constituting a preferred range within said greater range of 2 to 20 micrometers.

As can be appreciated, the intra-ocular implant 10 will have medical application not only as a corneal prosthesis but, as well, in a variety of post-operative and post-traumatic situations wherein less than permanent coverage of a portion of the eye is desirable. In such application, eventual removal of the implant may be readily facilitated through the use of laser means of a type now commonly used in ocular surgery, this without harm to tissue 20 from which growth of fibrous tissue into microgrooves 18 has occurred.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth herewith.

1. An intra-ocular implant, comprising:
an interior and a posterior surface and having an arcuate core element capable of optically transmitting light from an exterior of an eye to a retina thereof, said core formed of an alloplastic material, an arcuate first skirt extending integrally and annularly about said optical core, an outer skirt extending integrally and annularly about said first skirt in which said outer skirt is formed of an hydrophilic material capable of receiving vascular invasion and formation of fibrous tissue therein, said outer skirt further having a substantially flat posterior surface furnished with a multiplicity of microgrooves having respective widths in a range of between about two to about twenty micrometers and a depth within a like range of micrometers.

2. The implant as recited in claim 1, comprising:
a corneal prosthesis

3. The implant as recited in claim 1, comprising:
an apiketophakia prosthesis.
